(19) United States
(12) Patent Application Publication
(10) Pub. No.: US 2003/0232797 A1
(43) Pub. Date: Dec. 18, 2003

(54) Novel derivatives of androstane and androstene with ascorbic acid and use thereof in treating or preventing various conditions, diseases, and disorders

(75) Inventors: James P. Kutney, Vancouver (CA); Hongming Chen, Vancouver (CA); Duanjie Hou, Vancouver (CA); Changqing Wang, Vancouver (CA)

Correspondence Address: Susan M. Ben-Oliel, 2983 W. 41st. Ave, Vancouver, BC, V6N 3C8, CA

(73) Assignee: FORBES MEDI-TECH INC.

(21) Appl. No.: 10/165,769

(22) Filed: Jun. 6, 2002

(51) Int. Cl. C07J 17/00; A61K 31/585

(52) U.S. Cl. 514/175; 540/115

(57) ABSTRACT

The present invention provides novel derivatives comprising compounds in the androstane and androstene series, coupled with ascorbic acid, including salts thereof, and represented by one or more of the general formulae:

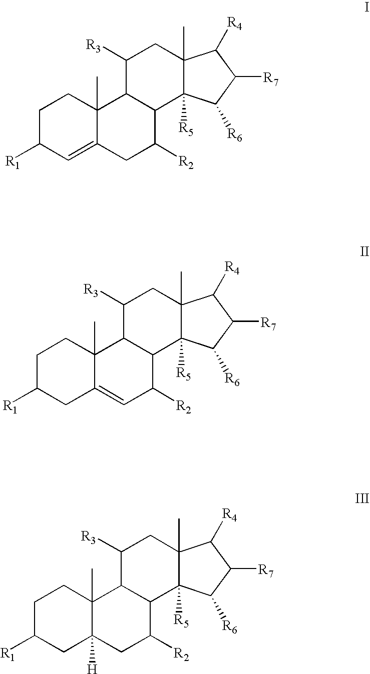

wherein R₁, R₂, R₃, R₄, R₅, R₆ may individually be chosen from hydrogen, OH, carbonyl, and an ascorbyl moiety; and R₇ may be hydrogen or any halogen.

### FIELD OF THE INVENTION

This present invention relates to the field of novel androstane and androstene steroid derivatives and the plurality of therapeutic uses of these derivatives.

### BACKGROUND OF THE INVENTION

The downstream metabolites of dehydroepiandrostrone (DHEA), particularly androstenediol (5-androstene-3β,17β-diol or AED) and androstenetriol (5-androstene-3β,7β,17β-triol or AET) have been well documented for their potential uses in the treatment of infectious diseases such as malaria and immune system disorders such as HIV, AIDS, hepatitis B and C. (1-3). These compounds also show protection against lethal radiation and restore immunity after radiation injury. (4-6). Furthermore, these compounds have been found to reduce the severity of ulcerative lesions and associated inflammation in rats with inflammatory bowel disease (7) and to enhance immune response leading to protection against bone loss in burn mice (8).

U.S. Pat. No. 5,559,107 to Gates and Loria describes esters and ethers of 5-androstene-3β,17β-diol and their use as regulators of immune response and cell proliferation and differentiation.

U.S. Pat. No. 5,206,008 to Loria describes esters and ethers of 5-androstene-3β,17β-diol and 5-androstene-3β,7β,17β-triol and their use in regulating immune response, ameliorating the effects of stress, and avoiding the negative effects of chemotherapy and irradiation exposure. Immune response regulation can be used as means to treat infectious diseases such as diabetes and chronic fatigue syndrome.

U.S. Pat. No. 5,296,481 to Partridge and Lardy provides aliphatic and aromatic esters of DHEA and their use in controlling weight gain and/or promoting weight loss without associated sex hormone synthesis.

U.S. Pat. No. 5,804,575 to Schwartz et al. teaches DHEA derivatives and their use as anti-cancer, anti-obesity, anti-diabetic and hypolipidemic agents. Related DHEA-derivative patents to Schwartz include U.S. Pat. Nos. 4,898,694; 5,001,119; 5,028,631; 5,157,031; 5,700,793; 5,714,481; and 5,744,462.

Ben-David, et al. (9) have observed that DHEA treatment has an anti-hypercholesterolemic effect in mice, while Coleman, et al. (10) report that administration of DHEA produces a marked hypoglycemic effect in C57BL/KsJ-db/db mice. The latter authors suggest that the therapeutic effect of DHEA might result from its metabolism to estrogens.

It is further known that DHEA and 16.alpha.-bromo-epiandrosterone are inhibitors of Epstein-Barr virus-induced transformation of human lymphocytes and that 16.alpha.-bromo-epiandrosterone is a more potent inhibitor of mammalian G6PDH than DHEA (11).

While DHEA has been found effective in the afore-described manners, there is however, evidence of an estrogenic effect after prolonged administration. DHEA is not an estrogen per se but is well known to be convertible into estrogens. In addition, the therapeutic dose of DHEA is rather high. It would therefore be highly desirable to provide steroids, which while having the same afore-described advantage of DHEA are more potent and do not produce an estrogenic effect.

Besides DHEA, other steroids are known in the art. The following patents are selected by way of example:

Great Britain Patent No. 989,503 to Burn, et al. discloses 6,16.beta.-dimethyl-3.beta.-hydroxyandrost-5-en-17-ones. These compounds are disclosed to be useful as possessing pituitary inhibiting action.

U.S. Pat. No. 2,833,793 to Dodson, et al. discloses 1.beta.,3.beta.-dihydroxy-5-androsten-17-one as an androgenic and anabolic agent.

U.S. Pat. No. 2,911,418 to Johns, et al. discloses 16.alpha.-chloro-3.beta.-hydroxyandrost-5-en-17-one and 3.beta.-hydroxy-16.alpha.-iodoandrost-5-en-17-one as an anti-androgen.

U.S. Pat. No. 3,148,198 discloses that 16.alpha.,16.beta.-difluoro-3.beta.-hydroxyandrost-5-en-17-one possess androgenic properties.

French Application No. FR-A 2,317,934 discloses the following compounds:

3 beta-hydroxy-16.epsilon.-methylandrost-5-en-17-one

3 beta-hydroxy-16.epsilon.-ethylandrost-5-en-17-one

3 beta-hydroxy-16.epsilon.-isopropylandrost-5-en-17-one

The Annual Report of the Fels Research Institute, pp. 32-33, (1979-1980) discloses the following compounds as having tumor-preventive, anti-obesity and anti-aging qualities:

3 beta-hydroxy-16.alpha.-bromo-5.alpha.-androstan-17-one

3 beta-hydroxy-16.alpha.-chloro-5.alpha.-androstan-17-one

3 beta-hydroxy-16.alpha.-fluoro-5.alpha.-androstan-17-one

3 beta-hydroxy-16.alpha.-iodo-5.alpha.-androstan-17-one

3 beta-hydroxy-16.alpha.-bromoandrost-5-en-17-one

16 alpha.bromoandrostan-17-one

Overall, DHEA and its metabolites are considered to be potent agents useful in a number of conditions and disorders, particularly as immunomodulating and anti-inflammatory compounds. More recently, the role of inflammation in cardiovascular disease (“CVD”) is becoming more understood. For example, Ricker et al. (12) describes a possible role of inflammation in the CVD process. J. Boyle (13) suggests an association between plaque rupture and atherosclerotic inflammation.

While recent advances in science and technology are helping to improve quality and add years to human life, the prevention of atherosclerosis, the underlying cause of cardiovascular disease (“CVD”) has not been sufficiently addressed. Atherosclerosis is a degenerative process resulting from an interplay of inherited (genetic) factors and environmental factors such as diet and lifestyle. Research to date suggest that cholesterol may play a role in atherosclerosis by forming atherosclerotic plaques in blood vessels, ultimately cutting off blood supply to the heart muscle or alternatively to the brain or limbs, depending on the location of the plaque in the arterial tree (14,15). Overviews have indicated that a 1% reduction in a person's total serum cholesterol yields a 2% reduction in risk of a coronary artery event (16). Statistically, a 10% decrease in average serum cholesterol (e.g. from 6.0 mmol/L to 5.3 mmol/L) may result in the prevention of 100,000 deaths in the United States annually (17).

One significant obstacle to the efficient use of the androstene and androstane family of compounds is their poor solubility. Accordingly, the provision of a stable, soluble compound which could be administered orally and which could be incorporated without further modification into delivery vehicles would be highly desirable and has not heretofore been satisfactorily achieved.

It is an object of the present invention to obviate or mitigate the above disadvantages.

### SUMMARY OF THE INVENTION

The present invention provides novel derivatives comprising compounds in the androstane and androstene series, coupled with ascorbic acid, including salts thereof, and represented by one or more of the general formulae:

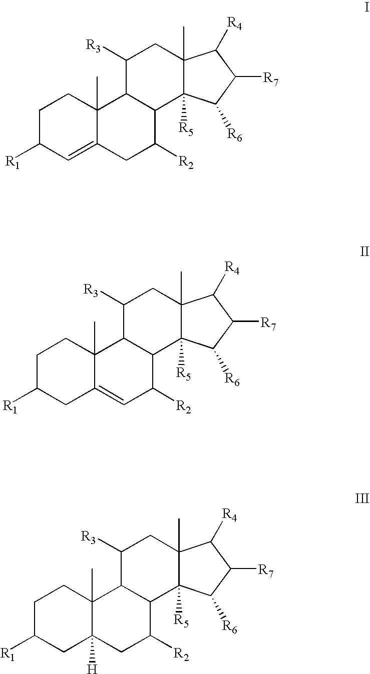

wherein R₁, R₂, R₃, R₄, R₅, R₆ may individually be chosen from hydrogen, OH, carbonyl, and an ascorbyl moiety; and R₇ may be hydrogen or any halogen.

The present invention also comprises processes of preparing the novel derivatives having the above noted formulae.

The present invention further comprises compositions for treating and/or preventing a plurality of diseases, conditions and disorders including, but not limited to, treating and/or preventing CVD and its underlying manifestations including atherosclerosis, hypercholesterolemia, hyperlipidemia, hypertension, thrombosis, coronary artery disease, aneurysm, myocardial infarction, embolism, stroke, thrombosis, angina or unstable angina, coronary plaque inflammation, related diseases such as Type II diabetes, as well as treating diseases, conditions or disorders in which immune function is compromised or in which immune system enhancement is required, including radiation-related injuries, HIV, AIDS, hepatitis, chronic fatigue syndrome, and malaria, as well as reducing inflammation, caused by, for example bacterial-induced inflammation, viral-induced inflammation, chronic inflammatory bowel disease and inflammation associated with surgical procedures and injury, as well as being useful to control weight gain or promote weight loss, as well as being useful in preventing cancer, as well as exhibiting anti-aging effects which comprise one or more derivatives or analogues of androstane and androstene coupled with ascorbic acid, having one or more of the above noted formulae, and a pharmaceutically acceptable or non-toxic food quality carrier therefor.

The present invention further provides foods, beverages and nutraceuticals supplemented with derivatives of androstane and/or androstene coupled with ascorbic acid, having one or more of the above noted formulae.

The present invention further provides a method for treating and/or preventing a plurality of diseases, conditions and disorders including, but not limited to, treating and/or preventing CVD and its underlying manifestations including atherosclerosis, hypercholesterolemia, hyperlipidemia, hypertension, thrombosis, coronary artery disease, aneurysm, myocardial infarction, embolism, stroke, thrombosis, angina or unstable angina, coronary plaque inflammation, related diseases such as Type II diabetes, as well as treating diseases, conditions or disorders in which immune function is compromised or in which immune system enhancement is required, including radiation-related injuries, HIV, AIDS, hepatitis, chronic fatigue syndrome, and malaria, as well as reducing inflammation, caused by, for example bacterial-induced inflammation, viral-induced inflammation, chronic inflammatory bowel disease and inflammation associated with surgical procedures and injury, as well as being useful to control weight gain or promote weight loss, as well as being useful in preventing cancer, as well as exhibiting anti-aging effects by administering to an animal, particularly a human, derivatives of androstane and/or androstene coupled with ascorbic acid, having one or more of the above noted formulae.

The androstane/androstene/ascorbic acid derivatives and salts thereof of the present invention have numerous advantages over non-modified compounds within the androstane/androstene family which are known and described in the art. In particular, it has been found that solubility in aqueous solutions such as water is improved thereby allowing oral administration per se and improving other modes of administration without any further enhancements or modifications. Accordingly, the derivatives of the present invention can be prepared and used as such or they can be easily incorporated into foods, beverages, pharmaceuticals and nutraceuticals regardless of whether these “vehicles” are water-based. This enhanced solubility generally translates into lower administration dosages of the derivatives in order to achieve the desired therapeutic effect.

These effects and other significant advantages are described in more detail below.

### BRIEF DESCRIPTION OF THE DRAWINGS

The present invention is illustrated by way the following non-limiting drawings in which:

FIG. 1 is a schematic showing the synthesis of one preferred derivative of the present invention, disodium ascorbyl phosphate ester of dehydroisoandrosterone;

FIG. 2 is a schematic showing the synthesis of one preferred derivative of the present invention, disodium ascorbyl phosphate ester of 5-Androstan-3-ol-17-one;

FIG. 3 is a schematic showing the synthesis of one preferred derivative of the present invention, disodium ascorbyl phosphate ester of Androst-5-ene-3,17-diol;

FIG. 4 is a schematic showing the synthesis of one preferred derivative of the present invention, disodium ascorbyl phosphate ester of Androst-5-ene-17-ol;

FIG. 5 is a schematic showing the synthesis of one preferred derivative of the present invention, tetra-sodium monoascorbyl diphosphate ester of 3-acetoxyandrost-5-ene-7,17-diol;

FIG. 6 is a schematic showing the synthesis of one preferred derivative of the present invention, tetrasodium diascorbyl diphosphate ester of Androst-5-ene-3,17-diol;

### PREFERRED EMBODIMENTS OF THE INVENTION

The following detailed description is provided to aid those skilled in the art in practising the invention. However this detailed description should not be construed so as to unduly limit the scope of the present invention. Modifications and variations to the embodiments discussed herein may be made by those with ordinary skill in the art without departing from the spirit or scope of the present invention.

According to the present invention, there are provided novel derivatives of androstene and/or androstane and ascorbic acid suitable for use per se in treating or preventing a wide variety of diseases, conditions and disorders.

The derivatives of the present invention are represented by one of the following core formulae:

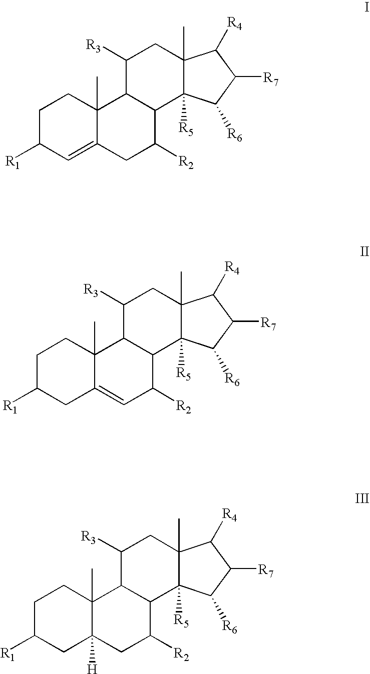

wherein R₁, R₂, R₃, R₄, R₅, R₆ may individually be chosen from hydrogen, OH, carbonyl, and an ascorbyl moiety, with at least one of these constituents being chosen as an ascorbyl moiety; and R₇ may be hydrogen or any halogen.

The components of the derivative will be described in more detail below. It should be noted that, throughout this disclosure, the terms “derivative”, “structure” and “analogue” are used interchangeably to describe the novel unitary compound which links or couples one of

the selected steroid moieties to ascorbic acid.

In a most preferred form of the present invention, the ascorbyl moiety which is coupled to the compound from the androstane or androstene family is selected individually from one or more of the following structures:

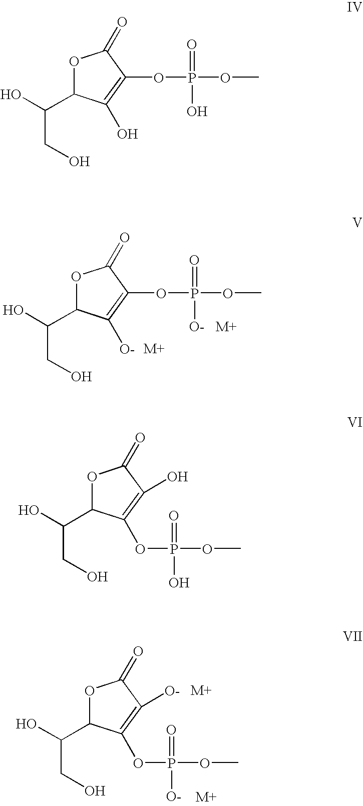

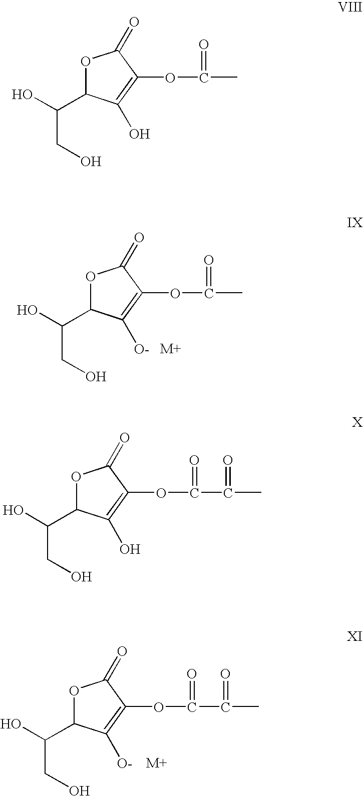

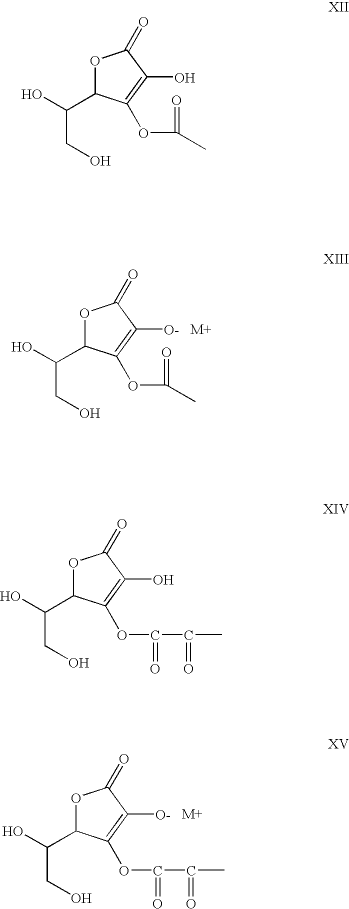

wherein M+ represents any metal, alkali earth metal, or alkali metal.

What is achieved within the scope of the present invention is the creation of a new structure or compound wherein an androstane or androstene moiety is chemically linked to ascorbic acid. The union benefits and enhances the both parts of this new structure. The steroid moiety, formerly poorly soluble, becomes, as part of the new derivative, much more readily soluble in aqueous and non-aqueous media such as oils and fats. Accordingly, administration of the steroid becomes possible without any further enhancements to modify its delivery.

For many years, it has been recognized that L-ascorbic acid (commonly known as vitamin C) is a vital part of balanced human nutrition and plays a role as a physiological anti-oxidant. However, ascorbic acid is the least stable vitamin with which to work since it reacts extremely easily with atmospheric oxygen yielding dehydroascorbic acid which further and readily decomposes into compounds void of vitamin C efficacy. It is believed that the new structure of the present invention “protects” ascorbic acid from such decomposition. Furthermore, it is believed that the anti-oxidative and other therapeutic effects of ascorbic acid are enhanced in a synergistic or additive fashion as a unitary compound formed with the androstane or androstene moieties. These advantages have not heretofore been appreciated or explored.

The most preferred derivatives of the present invention are represented by one or more of formulae I, II, and III noted above and the substituents R1-R7 are selected from one or more of the following combinations:

1) wherein R1 is an ascorbyl moiety, R2, R3, R5, R6 and R7 are H, and R4 is carbonyl;

2) wherein R1 is an ascorbyl moiety, R2, R3, R5 R6 and R7 are H, and R4 is OH;

3) wherein R4 is an ascorbyl moiety, R1 is OH, and R2, R3, R5, R6 and R7 are H;

4) wherein R4 is an ascorbyl moiety, R1 is carbonyl, and R2, R3, R5, R6 and R7 are H;

5) wherein R1 and R4 are ascorbyl moieties, and R2, R3, R5, R6, and R7 are H;

6) wherein R1 and R2 are ascorbyl moieties, R3, R5, R6 and R7 are H, and R4 is OH;

7) wherein R1 and R2 are ascorbyl moieties, R3, R5, R6, and R7 are H, and R4 is carbonyl;

8) wherein R1 and R4 are ascorbyl moieties, R2 is OH, and R3, R5, R6 and R7 are H;

9) wherein R3 is an ascorbyl moiety, R1 and R4 are carbonyl, and R2, R5, R6 and R7 are H;

10) wherein R3 is an ascorbyl moiety, R1 and R4 are OH, and R2, R5, R6 and R7 are H;

11) wherein R5 is an ascorbyl moiety, R1 and R4 are carbonyl, and R2, R3, R6 and R7 are H;

12) wherein R5 is an ascorbyl moiety, R1 and R4 are OH, and R2, R3, R6 and R7 are H;

13) wherein R6 is an ascorbyl moiety, R1 and R4 are carbonyl, and R2, R3, R5 and R7 are H;

14) wherein R6 is an ascorbyl moiety, R1 and R4 are OH, and R2, R3, R5 and R7 are H;

15) wherein R4 is an ascorbyl moiety, R1 and R2 are OH, and R3, R5, R6 and R7 are H;

16) wherein R4 is an ascorbyl moiety, R1 and R3 are OH, and R2, R5, R6 and R7 are H;

17) wherein R1 is an ascorbyl moiety, R3 and R4 are OH, and R2, R5, R6 and R7 are H;

18) wherein R1 is an ascorbyl moiety, R2 and R4 are OH, and R3, R5, R6 and R7 are H;

19) wherein R1, R2 and R4 are ascorbyl moieties, and R3, R5, R6 and R7 are H;

20) wherein R1 and R2 are ascorbyl moieties, R4 is carbonyl, and R3, R5, R6 and R7 are H;

21) wherein R1 is an ascorbyl moiety, R4 is carbonyl, R2, R3, R5,R6 are H, and R7 is a halogen;

22) wherein R1 and R4 are ascorbyl moieties, R2, R3, R5, R6 are H, and R7 is a halogen;

23) wherein R4 is an ascorbyl moiety, R1 is carbonyl, R2, R3, R5, R6 are H, and R7 is a halogen;

24) wherein R3 is an ascorbyl moiety, R4 is carbonyl, R1 is OH, R2, R5, R6 are H, and R7 is a halogen;

25) wherein R3 is an ascorbyl moiety, R4 is OH, R1 is carbonyl, R2, R5, R6 are H, and R7 is a halogen;

26) wherein R5 is an ascorbyl moiety, R1 and R4 are carbonyl, R2, R3, R6 are H, and R7 is a halogen;

27) wherein R5 is an ascorbyl moiety, R1 and R4 are OH, R2, R3, R6 are H, and R7 is a halogen;

28) wherein R6 is an ascorbyl moiety, R1 and R4 are carbonyl, R2, R3, R5 are H, and R7 is a halogen;

29) wherein R6 is an ascorbyl moiety, R1 and R4 are OH, R2, R3, R5 are H, and R7 is a halogen;

30) wherein R1, R3 and R4 are ascorbyl moieties, R2 and R5, R6 are H, and R7 is halogen;

31) wherein R1, R4 and R5 are ascorbyl moieties, R2 and R3, R6 are H, and R7 is halogen;

32) wherein R1 R2 and R4 are ascorbyl moieties, R3, R5, and R6 are H, and R7 is a halogen; and

33) wherein R1, R4, R6 are ascorbyl moieties; R2, R3, and R5 are H; and R7 is a halogen.

It is to be understood that these preferred derivatives include all biologically acceptable salts thereof. Halogens include chlorine (Cl), bromine (Br), fluorine (F) and iodine (I).

Derivative Formation

a) Ester Formation

There are many processes by which novel structures comprising compounds within the androstane and androstene family and ascorbic acid can be formed. In general, the selected steroid (or halophosphate, halocarbonate or halo-oxalate derivatives thereof) and ascorbic acid are mixed together under reaction conditions to permit condensation of the “acid” moiety with the “alcohol” (steroid). These conditions are the same as those used in other common esterification reactions such as the Fisher esterification process in which the acid component and the alcohol component are allowed to react directly or in the presence of a suitable acid catalyst such as mineral acid, sulfuric acid, phosphoric acid, p-toluenesulfonic acid. The organic solvents generally employed in such esterification reactions are ethers such as diethyl ether, tetrahydrofuran, or benzene, toluene or similar aromatic solvents and the temperatures can vary from room to elevated temperatures depending on the reactivity of the reactants undergoing the reaction.

In one preferred embodiment, the process to form the ester derivative comprises “protecting” the hydroxyl groups of the ascorbic acid or derivatives thereof as esters (for example, as acetate esters) or ethers (for example, methyl ethers) or cyclic ketals and then condensing the protected ascorbic acid with the steroid halophosphate, halocarbonate or halo-oxalate under suitable reaction conditions. In general, such condensation reactions are conducted in an organic solvent such as diethyl ether, tetrahydrofuran, or benzene, toluene or similar aromatic solvents. Depending on the nature and reactivity of the reactants, the reaction temperatures may vary from low (−15° C.) to elevated temperatures.

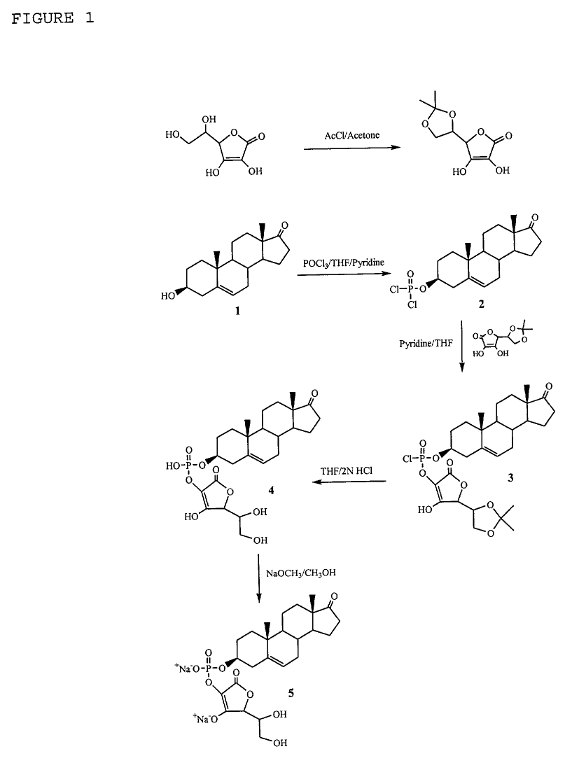

By way of example, FIG. 1 is a schematic showing the formation of the “protected” ascorbic acid (step a), the formation of the intermediary chlorophosphate/steroid derivative (step b), and the condensation reaction (steps c or d) yielding one of novel derivatives of the present invention.

In more detail, the process shown in FIG. 1 is as follows: ascorbic acid is initially converted to the cyclic ketal by the formation of 5,6-isopropylidene-ascorbic acid (shown above structure 2 in FIG. 1). This can be achieved by mixing acetone with ascorbic acid and an acid chloride under suitable reaction conditions (refer to Example 1 below). Dehydrosoandrosterone chlorophosphate is prepared by forming a solution of the steroid in anhydrous THS and pyridine (although other nitrogen bases such as aliphatic and aromatic amines may alternatively be used) and treating this solution with a phosphorus derivative such as phosphorus oxychloride. The latter suspension is then mixed with 5,6-isopropylidene-ascorbic acid in the presence of pyridine/THF at 0° C. to room temperature. Removal of the protecting group with HCL is accomplished at room temperature. After extraction, final washing and drying, the resultant novel product is ascorbyl phosphate ester of the selected steroid.

In another preferred form of the process of the present invention, ascorbic acid is protected at the hydroxyl sites not as 5,6-isopropylidene-ascorbic acid but as esters (for example as acetates, phosphates and the like.). The latter may then be condensed with the selected steroid, derivatized as described above, using known esterification methods ultimately to produce the structures of the present invention. The formation of mono and diphosphates of ascorbic acid is described thoroughly in the literature. For example, U.S. Pat. No. 4,939,128 to Kato et al., the contents of which are incorporated herein by reference, teaches the formation of phosphoric acid esters of ascorbic acid. Similarly, U.S. Pat. No. 4,999,437 to Dobler et al., the contents of which are also fully incorporated herein by reference, describes the preparation of ascorbic acid 2-phosphate. In Dobler et al., the core reaction of phosphorylating ascorbic acid or ascorbic acid derivatives with POCl3 in the presence of tertiary amines (described in German Laid Open Application DOS 2,719,303) is improved by adding to the reaction solution a magnesium compound, preferably an aqueous solution of a magnesium compound. Any of these known ascorbic acid derivatives can be used within the scope of the present invention.

b) Salt Formation

The present invention encompasses not only the parent structures comprising the selected steroid and ascorbic acid but also the salts thereof. These salts are even more water soluble than the corresponding parent compounds and therefore their efficacy and evaluation both in vitro and in vivo will be much improved.

Salt formation of the derivatives of the present invention can be readily performed by treatment of the parent compound with a series of bases (for example, sodium methoxide or other metal alkoxides) to produce the corresponding alkali metal salts. Other metal salts of calcium, magnesium, manganese, copper, zinc, and the like can be generated by reacting the parent with suitable metal alkoxides.

Derivatives

The present invention comprises all derivatives wherein compounds within the androstane and androstene family are coupled or linked with ascorbic acid, including all biologically acceptable salts thereof. The “linkage” between the steroid and ascorbyl moiety, thereby forming the ester, may take one or more forms as shown in structures IV to XV above.

Accordingly, the present invention comprises all phosphate, carbonate and oxalate/steroid/ascorbyl derivatives as shown in FIGS. 1 through 6 as structures 4 and 8 and including all intermediates in the formation of these derivatives. It is to be clearly understood; however, that these structures are only a selection of the many novel derivatives which fall within the purview of formulae 1, 11 and Ill. It is also to be understood that although sodium salts are shown as structures 5 and 9, other salts are included within the scope of the invention, as described above.

The present invention also comprises all halophosphate, halocarbonate and halooxalate/steroid/ascorbyl derivatives.

Uses and Advantages of Novel Steroid Analogues

In accordance with the present invention, it has been surprisingly discovered that the steroid derivatives described herein have enormous potential in various pharmacological fields while obviating many of the limitations of using these steroids alone. In particular, the present invention provides a method for treating and/or preventing a plurality of diseases, conditions and disorders including, but not limited to, treating and/or preventing CVD and its underlying manifestations including atherosclerosis, hypercholesterolemia, hyperlipidemia, hypertension, thrombosis, coronary artery disease, aneurysm, myocardial infarction, embolism, stroke, thrombosis, angina or unstable angina, coronary plaque inflammation, related diseases such as Type II diabetes, as well as treating diseases, conditions or disorders in which immune function is compromised or in which immune system enhancement is required, including radiation-related injuries, HIV, AIDS, hepatitis, chronic fatigue syndrome, and malaria, as well as reducing inflammation, caused by, for example bacterial-induced inflammation, viral-induced inflammation, chronic inflammatory bowel disease and inflammation associated with surgical procedures and injury, as well as being useful to control weight gain or promote weight loss, as well as being useful in preventing cancer, as well as exhibiting ant-aging effects, by administering to an animal, particularly a human, a therapeutically effective amount of one or more derivatives of androstane and/or androstene coupled with ascorbic acid, having the above noted formulae.

The term “therapeutically effective” is intended to qualify the amount of the compound(s) administered in order to achieve one or more of the following goals in animals, particularly humans:

1) to lower serum LDL cholesterol, to increase serum HDL cholesterol and/or to decrease serum triglycerides;

2) to modulate an immune response;

3) to reduce inflammation;

4) to modify viral, bacterial or parasitic activity;

5) to stimulate myelopoiesis;

6) to enhance resistance to bacterial, parasitic and/or viral infection;

7) to provide protection from radiation or to restore immunity after a radiation injury;

8) to control weight gain or promote weight loss;

9) to treat or manage symptoms of diabetes; and

10) to treat cancer.

The novel derivatives of the present invention, wherein ascorbic acid is attached to the androstane/androstene moiety affords many dietary and therapeutic advantages when compared to the use of steroids without such attachment. First and foremost, solubility of the novel derivatives is greatly enhanced, both in aqueous solutions and non-aqueous media such as oils and fats. With this greater solubility, effective dietary and therapeutic dosages and concomitantly costs, can be reduced. Secondly, it is possible that there is even a synergistic or at least an additive effect between the steroid moiety and the ascorbic acid, when united in one structure, in treating or preventing not only cardiovascular disease and its underlying conditions including atherosclerosis, hypercholesterolemia and hyperlipidemia but also in respect to diseases, conditions and disorders in which immune function is compromised or in which immune system enhancement is required, including radiation-related injuries, HIV, AIDS, hepatitis, chronic fatigue syndrome, and malaria, as well as reducing inflammation, caused by, for example bacterial-induced inflammation, viral-induced inflammation, chronic inflammatory bowel disease and inflammation associated with surgical procedures and injury. Thirdly, the formation of these derivatives allows the full potential of ascorbic acid to be realized while eliminating decomposition. Fourthly, these derivatives are heat stable (stable to oxidation and hydrolysis) which is essential for further processing in, for example, extruders and food processors.

Delivery Systems

Although it is fully contemplated within the scope of the present invention that the derivatives may be administered to animals, particularly humans, directly and without any further modification, it is possible to take further steps to enhance delivery and ensure even distribution throughout the food, beverage, pharmaceutical, nutraceutical and the like to which they are added. It is to be understood; however, that these steps are purely optional. Such enhancement may be achieved by a number of suitable means such as, for example, solubilizing or dispersing the derivatives to form emulsions, solutions and dispersions or self-emulsifying systems; lyophilizing, spray drying, controlled precipitating, or a combination thereof; forming solid dispersions, suspensions, hydrated lipid systems; forming inclusion complexations with cyclodextrins; and using hydrotopes and formulations with bile acids and their derivatives. Alternatively, and optionally in conjunction with any one of these solubility and/or dispersability enhancement methods, the derivatives may be incorporated into various vehicles in order to achieve the therapeutic objectives set out herein.

Without limiting the generality of the foregoing, the derivatives of the present invention may be admixed with various carriers or adjuvants to assist in direct administration or to assist in the incorporation of the composition into foods, beverages, nutraceuticals or pharmaceuticals. In order to appreciate the various possible vehicles of the delivery of the derivatives, the list below is provided. The doses of the derivatives will vary depending upon, among other factors, the disease, condition or disorder sought to be treated or prevented, the mode of delivery, the patient size and condition, the result to be achieved, as well as other factors known to those skilled in the art of food additives and medicinal agents.

1) Pharmaceutical Dosage Forms:

It is contemplated within the scope of the present invention that the derivatives of the present invention may be incorporated into various conventional pharmaceutical preparations and dosage forms such as tablets (plain and coated) for use orally, bucally or lingually, capsules (hard and soft, gelatin, with or without additional coatings) powders, granules (including effervescent granules), pellets, microparticulates, solutions (such as micellar, syrups, elixirs and drops), lozenges, pastilles, ampoules, emulsions, microemulsions, ointments, creams, suppositories, gels, transdermal patches and modified release dosage forms together with customary excipients and/or diluents and stabilizers.

The derivatives of the present invention, adapted into the appropriate dosage form as described above may be administered to animals, including humans, orally, by injection (intravenously, subcutaneously, intra-peritoneally, intra-dermally or intramuscularly), topically or in other ways.

The compounds of the present invention can be administered to a patient either by themselves, or in pharmaceutical compositions where they are mixed with suitable carriers or excipients.

Use of pharmaceutically acceptable carriers to formulate the compounds herein disclosed for the practice of the invention into dosages suitable for systemic administration is within the scope of the invention. With proper choice of carrier and suitable manufacturing practice, the compounds of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions, comprising one or more of the compounds of the present invention, include compositions wherein the active ingredients are contained in an effective amount to achieve their intended purpose. Determination of the effective amounts is well within the capability of those skilled in the art, especially in light of the detailed disclosure provided herein.

In addition to the active ingredients these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

2) Foods/Beverages/Nutraceuticals:

In another form of the present invention, the derivatives of the present invention may be incorporated into foods, beverages and nutraceuticals, including, without limitation, the following:

1) Dairy Products—such as cheeses, butter, milk and other dairy beverages, spreads and dairy mixes, ice cream and yoghurt;

2) Fat-Based Products—such as margarines, spreads, mayonnaise, shortenings, cooking and frying oils and dressings;

3) Cereal-Based Products—comprising grains (for example, bread and pastas) whether these goods are cooked, baked or otherwise processed;

4) Confectioneries—such as chocolate, candies, chewing gum, desserts, non-dairy toppings (for example Cool Whip™), sorbets, icings and other fillings;

5) Beverages—whether alcoholic or non-alcoholic and including colas and other soft drinks, juice drinks, dietary supplement and meal replacement drinks such as those sold under the trade-marks BOOSt™ and Ensure™; and

6) Miscellaneous Products—including eggs and egg products, processed foods such as soups, pre-prepared pasta sauces, pre-formed meals and the like.

The derivatives of the present invention may be incorporated directly and without further modification into the food, nutraceutical or beverage by techniques such as mixing, infusion, injection, blending, dispersing, emulsifying, immersion, spraying and kneading. Alternatively, the derivatives may be applied directly onto a food or into a beverage by the consumer prior to ingestion. These are simple and economical modes of delivery.

### EXAMPLES

The present invention is illustrated, but not limited, by the following examples:

### Example 1

### Protection of Ascorbic Acid and Synthesis of Disodium Ascorbyl Phosphate Ester of Dehydroisoandrosterone

To a dry round bottom flask, acetone (150 ml) and L-ascorbic acid (50 g) were added at 0° C. Acetyl chloride (7.5 ml) was added dropwise through an addition funnel in 10 minutes. The reaction mixture was stirred at 0° C. for 24 hours. The precipitate was filtered off and washed with acetone (3×20 ml). The white product, 5,6-isopropylidine ascorbic acid, was dried under vacuum for 1.5 hours to give a dry powder (52 g), yield 85%.

A dry three neck round bottom flask was fitted with a stirring bar, argon inlet and an addition funnel. A solution of dehydroisoandrosterone (FIG. 1, 1.73 g, 6 mmol) in anhydrous THF (15 ml) and pyridine (2.4 ml) was added dropwise to the mixture of anhydrous THF (12 ml) and POCl₃ (0.7 ml, 7.5 mmol) at 0° C. over a period of 10 minutes. A white precipitate formed immediately. The suspension was stirred at 0° C. for 40 minutes, and at room temperature for 1 hour and 40 minutes.

To the above suspension, a solution of 5,6-isopropylidine ascorbic acid (3.6 g, 16.67 mmol) in anhydrous pyridine (3 ml) and THF (30 ml) was added dropwise at 0° C. over a period of 20 minutes. The suspension was stirred at 0° C. for 30 minutes, and at room temperature for 1.5 hours. The formed pyridinium chloride was filtered out and washed with THF twice. The solvents were evaporated under reduced pressure at 40° C. to afford a residue (3, FIG. 1).

The residue (3, Scheme 1) was dissolved in THF (40 ml), and 2N HCl (30 ml) was added in one portion. The mixture was stirred at room temperature for 8 hours. THF was evaporated under a reduced pressure. The water layer was extracted with ethyl acetate (4×50 ml). The combined ethyl acetate solution was washed with brine (100 ml), and dried over Na₂SO₄. The solvent was evaporated to give a residue. The residue was dissolved in CHCl₃, and then hexanes was added to precipitate the product. The precipitated solid was filtered out, washed with hexanes and dried under vacuum (2.43 g, crude product, yield: 77%). The purification of phosphate ester was done by reverse phase C-18 chromatography (Waters, water/methanol=90/10 to 60/40). Pure compound 4 (FIG. 1, 39 mg) was isolated from 50 mg of the crude product. The overall yield (base on dehydroisoandrosterone) was 60%.

Ascorbyl phosphate ester of dehydroisoandrosterone (4, Scheme 1, 0.5 g, 0.95 mmol) was dissolved in methanol (3 ml) at room temperature, and then sodium methoxide in methanol (1 ml, 20%) was added. The suspension was stirred at room temperature for 30 minutes. The precipitated solid was filtered out, washed with methanol, acetone and hexanes. The mother liquor was concentrated to 2 ml, acetone was added to precipitate the product. An additional white solid was obtained. The combined solid was dried under vacuum at room temperature. Disodium ascorbyl phosphate ester of dehydroisoandrosterone (5, FIG. 1, 0.49 g, yield 91%) was obtained.

### Example 2

### Synthesis of Disodium Ascorbyl Phosphate Ester of 5α-Androstan-3β-ol-17-one

To a dry round bottom flask, 5α-androstan-3β-ol-17-one (1.0 g, 3.4 mmol), THF (8.6 ml) and pyridine (1.38 ml) were added. The mixture was stirred at room temperature until a clear solution was obtained. To another dry round bottom flask, THF (6.9 ml) and POCl₃ (0.4 ml, 4.25 mmol) were added, stirred at 0° C. for 5 minutes. To this mixture, the above prepared 5α-androstan-3β-ol-17-one solution was added drop-wise under argon atmosphere over a period of 10 minutes. After the addition, the white suspension was stirred at 0° C. for 35 minutes, and at room temperature for 2 hours. The reaction was stopped and the white suspension was used for the coupling reaction without filtration.

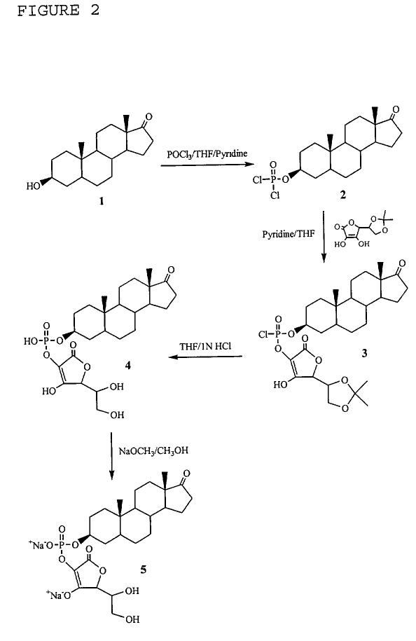

5,6-Isopropylidine ascorbic acid (2.0 g, 9.52 mmol) was dissolved in pyridine (1.71 ml) and THF (17 ml). The round bottom flask which contained previously prepared white suspension (2, FIG. 2) was immersed in an ice-water bath. To this mixture, the above prepared THF solution of the 5,6-isopropylidine ascorbic acid was added dropwise under stirring at 0° C. over a period of 15 minutes. After the addition, the mixture was stirred at 0° C. for 25 minutes, and at room temperature for 2 hours. The white solid of pyridinium chloride was filtered out and washed with THF (8 ml). The filtrate was concentrated to remove THF and excess pyridine to give a residue (3, FIG. 2, 2.38 g). The residue (3, Figure) was dissolved in THF (30 ml), and 1 N HCl (30 ml) was added in one portion. The mixture was stirred at room temperature for 16 hours and 45 minutes. 12N HCl (4 ml) was added to the reaction mixture at room temperature. The reaction mixture was stirred at room temperature for an additional 4 hours and 45 minutes. THF was evaporated under a reduced pressure. The water layer was extracted with ethyl acetate (3×60 ml). The combined ethyl acetate solution was washed with brine (60 ml), and dried over Na₂SO₄. The extract was concentrated to about 3 ml. Hexanes (15 ml) was added to precipitate the product. The precipitated solid was filtered out, washed with hexanes and dried under a reduced pressure (1.48 g, 4, FIG. 2).

Ascorbyl phosphate ester of 5α-androstan-3β-ol-17-one (4, FIG. 2, 0.5 g, 0.95 mmol) was dissolved in methanol (3 ml) at room temperature, and then sodium methoxide in methanol (1.5 ml, 20%) was added. The suspension was stirred at room temperature for 25 minutes. The precipitated solid was filtered out, washed with methanol, acetone and hexanes. The mother liquid was concentrated to 2 ml, and then acetone was added to precipitate the product. An additional product was obtained. The combined solid was dried under a reduced pressure at room temperature to give disodium ascorbyl phosphate ester of 5α-androstan-3β-ol-17-one (5, FIG. 2, 0.38 g). The overall yield was 57% (based on 5α-androstan-3β-ol-17-one).

### Example 3

### Synthesis of Disodium Ascorbyl Phosphate Ester of Androst-5-ene-3β,17β-diol

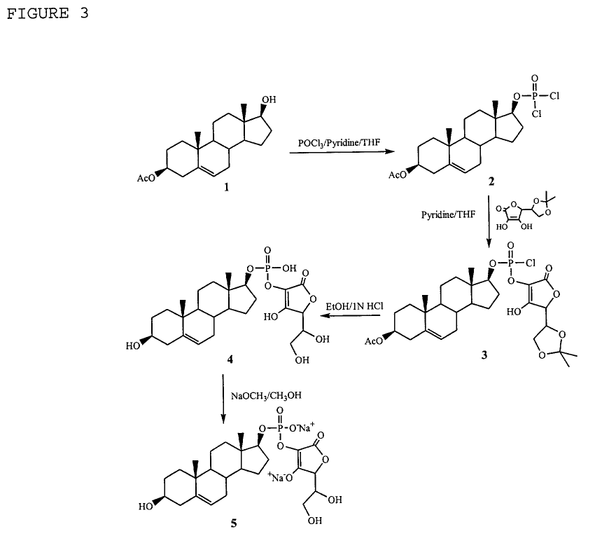

To a dry round bottom flask, 3β-acetoxyandrost-5-ene-17β-ol (1, FIG. 3, 1.0 g, 3.0 mmol), anhydrous THF (6.3 ml) and pyridine (0.73 ml) were added. The mixture was stirred at room temperature until a clear solution was obtained. To another dry round bottom flask, THF (2 ml) and POCl₃ (0.35 ml, 3.22 mmol) were added, stirred at −5° C.˜−10° C. for 5 minutes. To this mixture, the above prepared 3β-acetoxyandrost-5-ene-17β-ol solution was added drop-wise under argon atmosphere over a period of 20 minutes. After the addition, the white suspension was stirred at room temperature for 1 hour. The mixture was concentrated to remove THF and excess POCl₃ to give a residue (2, FIG. 3).

5,6-Isopropylidine ascorbic acid (0.98 g, 4.55 mmol) was dissolved in anhydrous pyridine (0.70 ml) and THF (6.2 ml). The residue (2, FIG. 3 dissolved in dry THF (4 ml). To this mixture, the above prepared THF solution of the 5,6-isopropylidine ascorbic acid added dropwise under stirring at 0° C. over a period of 20 minutes. After the addition, the mixture was stirred at room temperature for 1 hour and 25 minutes. The white solid of pyridinium chloride was filtered out and washed with THF (6 ml). The filtrate was concentrated to remove THF and excess pyridine to give a residue (3, FIG. 3).

The residue (3, FIG. 3) was dissolved in a mixture of ethanol (12.5 ml) and 1N HCl (12.5 ml). The mixture was kept stirring at 50° C. 55° C. for additional 3 hours and 45 minutes (TLC monitoring). The mixture was extracted with ethyl acetate (60 ml), washed with 10% aqueous NaCl twice (30 ml, 20 ml) and dried over Na₂SO₄ (10 g) for 1.5 hours. After the filtration, the filtrate was concentrated to 5 ml. Hexanes (10 ml) was added to precipitate the product. The precipitate was collected, washed with hexanes (10 ml) and dried under the reduced pressure to give a slightly yellow powder (4, FIG. 3, 0.95 g, crude product, yield 60%). The pure product was obtained by preparative HPLC.

Instrument is Waters Delta Preparative 4000 HPLC system. Column is Waters Symmetry C18, 5 μm, 30×100 mm. Mobile phases are 0.1% H₃PO₄ in water and acetonitrile. Water and acetonitrile are HPLC grade or equivalent.

The crude product was purified by preparative HPLC. The product was collected and evaporated on a rotary evaporator to remove acetonitrile. The water solution was extracted with ethyl acetate twice. The ethyl acetate layer was dried over Na₂SO₄, concentrated and dried under a reduced pressure to give a white powder product. This product was submitted for NMR and mass spectra. Both spectra indicated the product is ascorbyl phosphate ester of androst-5-ene-3β,17β-diol (4, Figure).

Preparation of disodium ascorbyl phosphate ester of androst-5-ene-3β,17β-diol (5, FIG. 3) was similar to the process described in Example 2.

### Example 4

### Synthesis of Disodium Ascorbyl Phosphate Ester of Androst-5-ene-17β-ol

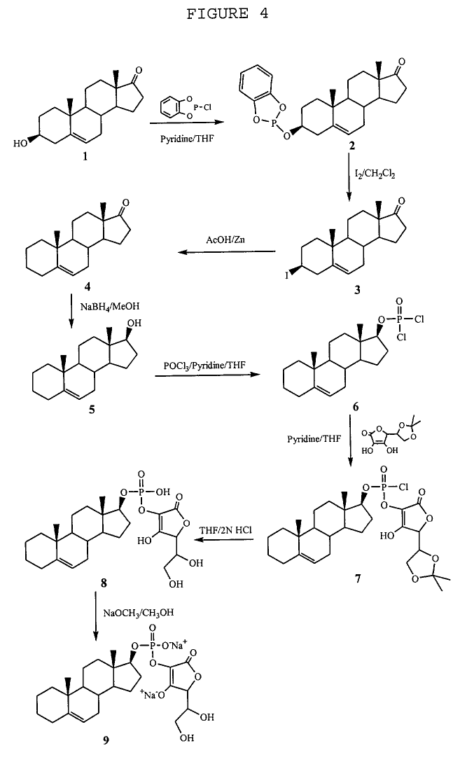

To a solution of pyridine (0.41 ml) and 1,2-phenylenephosphorochloridite (0.6 ml, 5 mmol) in anhydrous THF (10 ml) at 0° C. was added dropwise dehydroisoandrosterone (1, FIG. 4, 1.44 g, 5 mmol) in anhydrous THF (10 ml) over a period of 10 minutes. The reaction mixture was stirred at 0° C. for 30 minutes, and at room temperature for 4 hours. The reaction was monitored with TLC (hexanes/EtOAc=2/1). The formed pyridinium chloride was filtered off and washed with THF. The solvents were evaporated at 40° C. to give a white powder (2, FIG. 4).

The crude phosphite ester (2, FIG. 4) was dissolved in methylene chloride (25 ml), and treated with iodine (1.27 g) for 4 hours at room temperature. The reaction mixture was diluted with methylene chloride (75 ml), washed with 1N NaOH (2×50 ml) and water (2×50 ml), and dried over Na₂SO₄. The solvent was removed, and the product (3, Scheme 4, 1.4 g, yield 71%) was crystallized from methylene chloride and methanol.

3β-Iodoandrost-5-ene-17-one (3, FIG. 4, 1.27 g, 3.19 mmol) was dissolved in glacial acetic acid (40 ml) at 50-55° C., the activated zinc dust (2.7 g) was added in one portion. The mixture was stirred at 50° C.˜55° C. for 2 hours, the zinc dust was filtered out and washed with methylene chloride. The solution was diluted with methylene chloride (120 ml), washed with water (2×100 ml), 1N NaOH (2×100 ml) and water (100 ml), and dried over Na₂SO₄. The solvent was removed to afford a white powder. The white powder was dried under vacuum to give androst-5-ene-17-one (4, FIG. 4, 0.83 g, yield: 95%).

Androst-5-ene-17-one (4, FIG. 4, 0.65 g, 2.34 mmol) was dissolved in methanol (25 ml) at room temperature. The solution was cooled down to 0° C., and NaBH₄ (50 mg) was added in one portion. The mixture was stirred at 0° C. for 3 hours, and monitored with TLC (hexanes/EtOAc=3/1). After 3 hours, another portion of NaBH₄ (20 mg) was added, and the reaction mixture was stirred at 0° C. for additional half an hour. Aqueous NH₄Cl (5%, 25 ml) and HCl (6N, 5 ml) were added slowly at 0° C., and stirred for 1 hour. Water (100 ml) was added to completely precipitate the product. The precipitated solid was filtered out and washed with water, and dried under vacuum. The pure product (5, FIG. 4, 0.62 g, yield: 95%) was obtained by column chromatography.

A solution of androst-5-ene-17β-ol (5, FIG. 4, 0.63 g, 2.3 mmol) in anhydrous THF (8 ml) and pyridine (1 ml) was added drop-wise to the mixture of anhydrous THF (6 ml) and POCl₃ (0.28 ml, 3 mmol) at 0° C. over a period of 5 minutes. The suspension was stirred at 0° C. for 50 minutes, and then at room temperature for one hour (6, FIG. 4).

To the above suspension, a solution of 5,6-isopropylidine ascorbic acid (1.38 g) in anhydrous pyridine (1.2 ml) and THF (12 ml) was added drop-wise at 0° C. over a period of 15 minutes. The suspension was stirred for 1.5 hours at 0° C., and then overnight at room temperature. The formed pyridine hydrochloride was filtered out and washed with THF twice. The solvents were evaporated under reduced pressure at 40° C. to afford a residue (7, FIG. 4).

The residue (7, FIG. 4) was dissolved in THF (35 ml), and 2N HCl (30 ml) was added as one portion. The mixture was stirred overnight at room temperature. THF was evaporated under reduced pressure. The water layer was extracted with ethyl acetate (3×100 ml). The combined ethyl acetate solution was washed with brine (100 ml), and dried over Na₂SO₄. The solvent was evaporated to give a residue. The residue was dissolved in acetone, and hexanes was added to precipitate the product. The white precipitated solid was filtered out, washed with hexanes and dried under vacuum (8, FIG. 4, 0.82 g, crude product, yield: 70%).

Preparation of disodium ascorbyl phosphate ester of androst-5-ene-17β-ol was similar to example 1.

### Example 5

### Synthesis of Tetra-Sodium Monoascorbyl Diphosphate Ester of 3-Acetoxyandrost-5-ene-7β,17β-diol

To a dry round bottom flask, 3β-acetoxyandrost-5-ene-7β,17β-diol (0.5 g, 1.43 mmol), pyridine (0.83 ml) and THF (4 ml) were added. The mixture was stirred at room temperature until a clear solution was obtained. To another dry round bottom flask, THF (5 ml) and POCl₃ (0.33 ml) were added, stirred at −5° C, O° C. for 5 minutes. To this mixture, the above prepared 3β-acetoxyandrost-5-ene-7β,17β-diol solution was added dropwise under argon atmosphere over a period of 15 minutes. After the addition, the white suspension was stirred at room temperature for 2 hours and 45 minutes. The reaction was stopped and the white suspension was used for the coupling reaction without filtration.

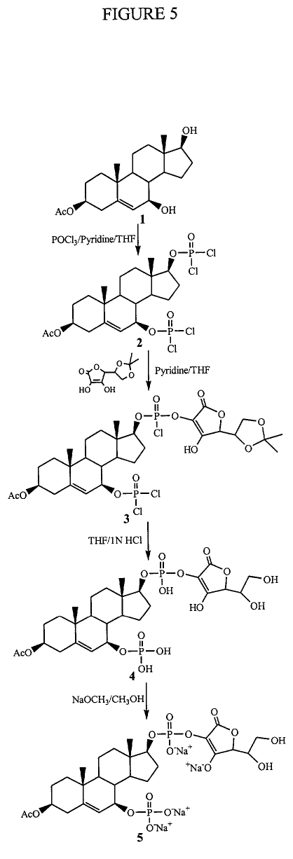

5,6-Isopropylidine ascorbic acid (1.30 g, 6.02 mmol) was dissolved in pyridine (1.16 ml) and THF (5.8 ml). The round bottom flask which contained previously prepared white suspension (2, FIG. 5) was immersed in an ice-water bath. To this mixture, the above prepared THF solution of the 5,6-isopropylidine ascorbic acid was added dropwise under stirring at 0° C. over a period of 15 minutes. After the addition, the mixture was stirred at 0° C. for 40 min and at room temperature for 17 hours. The white solid of pyridinium chloride was filtered out and washed with THF (5 ml). The filtrate was concentrated to remove THF and excess pyridine to give a residue (3, FIG. 5, 2.76 g). The crude of compound 3 (FIG. 5) was dissolved in a mixture of THF (30 ml) and 1N HCl (30 ml). The mixture was kept stirring at room temperature for 3.5 hours (TLC monitoring). The second portion of 1 N HCl (10 ml) were added. The mixture was stirred for an additional 18.5 hours. The THF in the reaction mixture was removed under a reduced pressure. The water suspension was extracted with ethyl acetate and n-butanol (1:1, 110 ml). The organic layer was washed with distilled water (11 ml). The organic layer was concentrated on a rotary evaporator to give a residue. This residue was washed with hexanes (2×10 ml) and dried under the reduced pressure to give a crude product (4, FIG. 5, 1.15 g).

Preparation of sodium salt of compound 4 (FIG. 5) was similar to Example 2.

### Example 6

### Synthesis of Tetrasodium Diascorbyl Diphosphate Ester of Androst-5-ene-3β,17β-diol

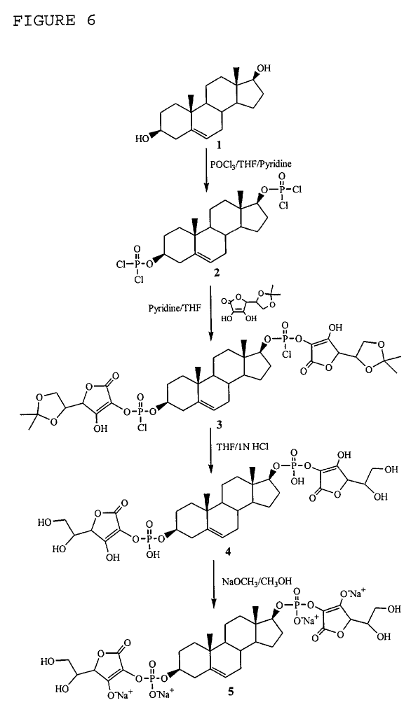

In a dry round bottom flask, androst-5-ene-3β,17β-diol (1, FIG. 6, 1.5 g, 5.17 mmol) was dissolved in pyridine (3.0 ml) and THF (15 ml). Into another dry round bottom flask was added THF (20 ml) and POCl₃ (1.17 ml, 12.56 mmol). The latter was stirirred at −5° C. for 5 minutes before the addition of androst-5-ene-3β,17β-diol (1, FIG. 6) over a period of 20 minutes. White precipitate was observed shortly after the addition of 1 (FIG. 6), and after the initial 20 minutes of reaction at −5° C., the reaction was allowed to continue at room temperature for 2.5 hours.

The flask was then cooled to 0° C., and a solution of 5,6-isopropylidene ascorbic acid (3.19 g, 14.78 mmol) in pyridine (3 ml) and THF (15 ml) was added drop-wise over a period of 20 minutes under vigorous stirring. The reaction was allowed to continue for another two hours. Then, the reaction mixture was filtered, and the filtrate was concentrated to a thick syrup. Heptane was added and the mixture was distilled under a reduced pressure. A solid crude 3 (FIG. 6) was obtained.

The crude 3 (FIG. 6) was dissolved in THF/1 N HCl (1:1, 150 ml), and the hydrolysis was carried out at room temperature under vigorous stirring. After 12 hours of reaction, a TLC test indicated that the hydrolysis was complete. The THF in the reaction mixture was removed under a reduced pressure at room temperature, and n-butanol and ethyl acetate (1:1, 100 ml) was used for the extraction. The organic layer was washed with water (2×20 ml), and then concentrated to afford the crude product of diascorbyl diphosphate ester of androst-5-ene-3β,17β-diol (4, FIG. 6, 3.0 g).

The crude diascorbyl diphosphate ester of androst-5-ene-3,7 17β-diol (4, FIG. 6, 400 mg) was dissolved in methanol (5 ml). To this solution was added 2 ml of sodium methoxide in methanol (20%, w/v) under magnetic stirring. White precipitate was observed upon the addition of sodium methoxide methanol solution. The suspension was stirred for half an hour before it was filtered and washed with methanol and acetone. The solid product was dried under high vacuum, and tetrasodium diascorbyl diphosphate ester of androst-5-ene-3β,17β-diol (5, FIG. 6, 330 mg) was obtained.

### Example 7

### Solubility Data

Selected derivatives formed in accordance with the present invention were tested for solubility using the following protocol: Into an 1 ml glass vial was added 50 mg of the sample to be tested. Water (or other desired solvent) was added portion by portion (50 micro liter per portion) at an interval of 10 minutes until a clear solution was obtained. An ultrasonic bath was employed to enhance the solubilizing process. The weight of the water

added was determined by an analytical balance. The solubility was thus obtained by the following calculation: Solubility (% w/w)=50/(50+weight of water in mg).

| | Chemical name, molecular | |
|---|---|---|
| Structures | formula & formula weight | Solubility |
| 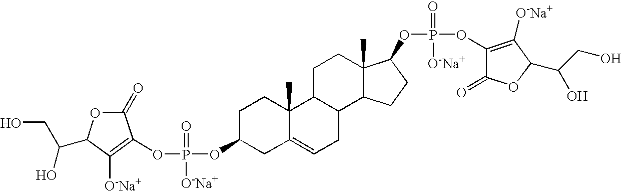 | Diascorbyl diphosphate of androst-5ene-3β,17β-diol, tetrasodium salt C₃₁H₄₀Na₄O₁₈P₂F.W 854.55 | Soluble in water (10.6%, w/w) Slightly soluble in ethanol |
| 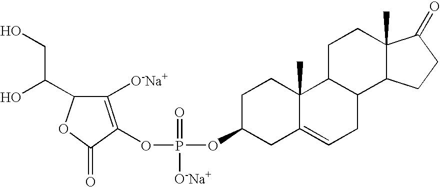 | Ascorbyl phosphate of dehydroisoandrosterone, disodium salt C₂₅H₃₃Na₂O₁₀P F.W 570.48 | Soluble in water (10.1%, w/w) Slightly soluble in ethanol |
| 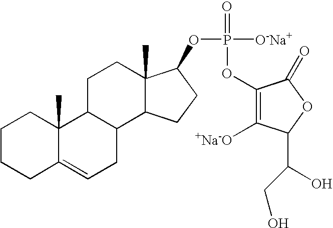 | Ascorbyl phosphate of androst-5ene-17β-ol, disodium salt C₂₅H₃₅Na₂O₉P F.W 556.49 | Soluble in water (9.6%, w/w) Slightly soluble in ethanol |
| 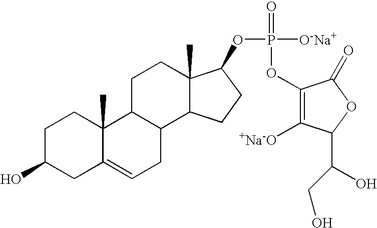 | Ascorbyl phosphate of androst-5-ene-3β,17β-diol, Disodium salt C₂₅H₃₅Na₂O₁₀P F.W 572.49 | Soluble in water (9.0%, w/w) Slightly soluble in ethanol |
| 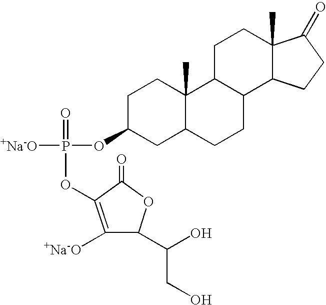 | Ascorbyl phosphate of 5α-androststan-3β-ol-17- one, Disodium salt C₂₅H₃₅Na₂O₁₀P F.W 572.49 | Soluble in water (9.5%, w/w) Slightly soluble in ethanol |

### REFERENCES

1. 50^{th} American Society of Tropical Medicine and Hygiene, Atlanta Ga., Nov. 12, 2001, Frincke et. Al. Clinical Efficacy of HE2000, An Androstane Immune Regulating Hormone

2. The Cytokine Odyssey 2001; Society for Leukocyte Biology and the International Cytokine Society, Reading et al. Elevated Inflammation-Related Transcripts in HIV-Infected Individuals Nov. 8-11, 2001

3 4^{th} International Workshop on HIV Cells of Macrophage Lineage and other Resevoirs Dec. 1-4, 1999, Hollis-Eden Pharmaceuticals Inc Abstract on HE2000 Drug Administration, Reading et al.

4. Neuroimmunomodulation, volume 197 of the Annals of the New York Academy of Sciences, Loria et al., page 860-867

5. Radiation Research 2001; 156: 283-293

6. International Journal of Immunopharmacology 2000;22:1-14

7. The Cytokine Odyssey 2001; Society for Leukocyte Biology and the International Cytokine Society, Richard et al. Effects of Androstenetriol on Microscopic Lesions of Ulcerative Colitis in rat Model of Inflammatory Bowel Disease, Nov. 8-11, 2001

8. The Cytokine Odyssey 2001; Society for Leukocyte Biology and the International Cytokine Society, Ahlem et al. Administration of beta-AET to burn mice modifies bone loss, Nov. 8-11, 2001

9. Anti-hypercholesterolemic effect of dehydroepiandrosterone in rats, Proc. Soc. Expt. Biol. Med., 125, 1136-1140 (1967)

10 Diabetes 1982;31: 830

11. Schwartz, et al. Carcinogensis, Vol. 2 No. 7, 683-686 (1981).

12 New Eng. J. Med. 1999; 336:973-9

13 J. Path. 1997; 181: 93-99

14. Law M R, Wald N J, Wu, Hacksaw Z A, Bailey A: Systemic underestimation of association between serum cholesterol concentration and ischemic heart disease in observational studies: Data from BUPA Study; Br. Med. J. 1994; 308: 363-366

15. Law M R, Wald N J, Thompson S G: By how much and how quickly does reduction in serum cholesterol concentration lower risk of ischemic heart disease? Br. Med. J. 1994; 367-373

16. La Rosa J.C., Hunninghake D. Bush D. et al.; The cholesterol facts: A summary of the evidence relating to dietary fats, serum cholesterol and coronary heart disease:Ajoint statement by the American Heart Association and the National Heart, Lung and Blood Institute. Circulation 1990; 81:1721-1733

17. Havel R. J., Rapaport E. Drug Therapy: Management of Primary Hyperlipidemia. New England Journal of Medicine, 1995; 332:1491-1498

We claim:

1. A derivative comprising compounds in the androstane and androstene series, coupled with ascorbic acid, including salts thereof, and represented by one or more of the general formulae: 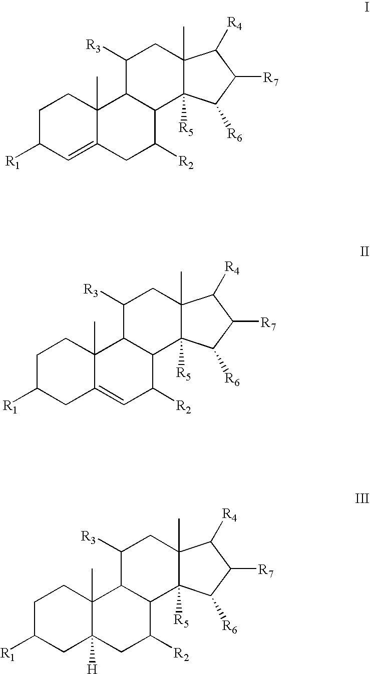
wherein R₁, R₂, R₃, R₄, R₅, R₆ may individually be chosen from hydrogen, OH, carbonyl, and an ascorbyl moiety; and R₇ may be hydrogen or any halogen.

2. The derivative of claim 1 wherein the ascorbyl moiety is: 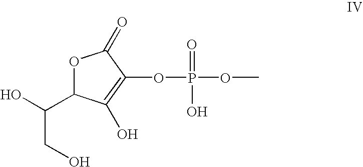
or one of its' biologically acceptable salts.

3. The derivative of claim 1 wherein the ascorbyl moiety is: 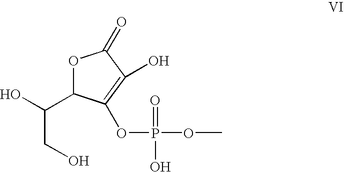
or one of its' biologically acceptable salts.

4. The derivative of claim 1 wherein the ascorbyl moiety is: 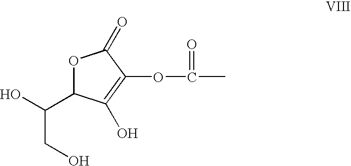
or one of its' biologically acceptable salts.

5. The derivative of claim 1 wherein the ascorbyl moiety is: 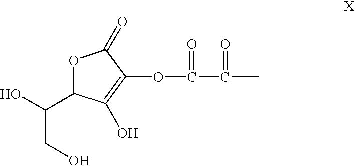
or one of its' biologically acceptable salts.

6. The derivative of claim 1 wherein the ascorbyl moiety is: 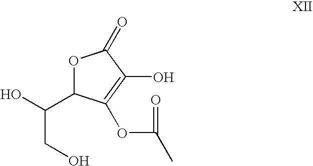
or one of its' biologically acceptable salts.

7. The derivative of claim 1 wherein the ascorbyl moiety is: 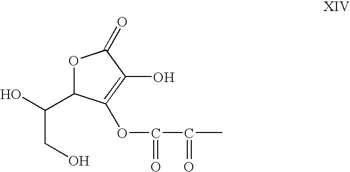
or one of its' biologically acceptable salts.

8. The derivative of claim 1 wherein R1 is an ascorbyl moiety, R2, R3, R5, R6 and R7 are H, and R4 is carbonyl.

9. The derivative of claim 1 wherein R1 is an ascorbyl moiety, R2, R3, R5 R6 and R7 are H, and R4 is OH.

10. The derivative of claim 1 wherein R4 is an ascorbyl moiety, R1 is OH, and R2, R3, R5, R6 and R7 are H.

11. The derivative of claim 1 wherein R4 is an ascorbyl moiety, R1 is carbonyl, and R2, R3, R5, R6 and R7 are H.

12. The derivative of claim 1 wherein R1 and R4 are ascorbyl moieties, and R2, R3, R5, R6, and R7 are H.

13. The derivative of claim 1 wherein R1 and R2 are ascorbyl moieties, R3, R5, R6 and R7 are H, and R4 is OH.

14. The derivative of claim 1 wherein R1 and R2 are ascorbyl moieties, R3, R5, R6, and R7 are H, and R4 is carbonyl.

15. The derivative of claim 1 wherein R1 and R4 are ascorbyl moieties, R2 is OH, and R3, R5, R6 and R7 are H.

16. The derivative of claim 1 wherein R3 is an ascorbyl moiety, R1 and R4 are carbonyl, and R2, R5, R6 and R7 are H.

17. The derivative of claim 1 wherein R3 is an ascorbyl moiety, R1 and R4 are OH, and R2, R5, R6 and R7 are H.

18. The derivative of claim 1 wherein R5 is an ascorbyl moiety, R1 and R4 are carbonyl, and R2, R3, R6 and R7 are H.

19. The derivative of claim 1 wherein R5 is an ascorbyl moiety, R1 and R4 are OH, and R2, R3, R6 and R7 are H.

20. The derivative of claim 1 wherein R6 is an ascorbyl moiety, R1 and R4 are carbonyl, and R2, R3, R5 and R7 are H.

21. The derivative of claim 1 wherein R6 is an ascorbyl moiety, R1 and R4 are OH, and R2, R3, R5 and R7 are H.

22. The derivative of claim 1 wherein R4 is an ascorbyl moiety, R1 and R2 are OH, and R3, R5, R6 and R7 are H.

23. The derivative of claim 1 wherein R4 is an ascorbyl moiety, R1 and R3 are OH, and R2, R5, R6 and R7 are H.

24. The derivative of claim 1 wherein R1 is an ascorbyl moiety, R3 and R4 are OH, and R2, R5, R6 and R7 are H.

25. The derivative of claim 1 wherein R1 is an ascorbyl moiety, R2 and R4 are OH, and R3, R5, R6 and R7 are H.

26. The derivative of claim 1 wherein R1, R2 and R4 are ascorbyl moieties, and R3, R5, R6 and R7 are H.

27. The derivative of claim 1 wherein R1 and R2 are ascorbyl moieties, R4 is carbonyl, and R3, R5, R6 and R7 are H.

28. The derivative of claim 1 wherein R1 is an ascorbyl moiety, R4 is carbonyl, R2, R3, R5,R6 are H, and R7 is a halogen.

29. The derivative of claim 1 wherein R1 and R4 are ascorbyl moieties, R2, R3, R5, R6 are H, and R7 is a halogen.

30. The derivative of claim 1 wherein R4 is an ascorbyl moiety, R1 is carbonyl, R2, R3, R5, R6 are H, and R7 is a halogen.

31. The derivative of claim 1 wherein R3 is an ascorbyl moiety, R4 is carbonyl, R1 is OH, R2, R5, R6 are H, and R7 is a halogen.

32. The derivative of claim 1 wherein R3 is an ascorbyl moiety, R4 is OH, R1 is carbonyl, R2, R5, R6 are H, and R7 is a halogen.

33. The derivative of claim 1 wherein R5 is an ascorbyl moiety, R1 and R4 are carbonyl, R2, R3, R6 are H, and R7 is a halogen.

34. The derivative of claim 1 wherein R5 is an ascorbyl moiety, R1 and R4 are OH, R2, R3, R6 are H, and R7 is a halogen.

35. The derivative of claim 1 wherein R6 is an ascorbyl moiety, R1 and R4 are carbonyl, R2, R3, R5 are H, and R7 is a halogen.

36. The derivative of claim 1 wherein R6 is an ascorbyl moiety, R1 and R4 are OH, R2, R3, R5 are H, and R7 is a halogen.

37. The derivative of claim 1 wherein R1, R3 and R4 are ascorbyl moieties, R2 and R5, R6 are H, and R7 is a halogen.

38. The derivative of claim 1 wherein R1, R4 and R5 are ascorbyl moieties, R2 and R3, R6 are H, and R7 is a halogen.

39. The derivative of claim 1 wherein R1, R2 and R4 are ascorbyl moieties, R3 and R5, R6 are H, and R7 is a halogen.

40. The derivative of claim 1 wherein R1, R4, R6 are ascorbyl moieties; R2, R3, and R5 are H; and R7 is a halogen.

41. A method of enhancing immune response in an animal by the administration of an immune-enhancing effective amount of a derivative having one or more of the following formulae: 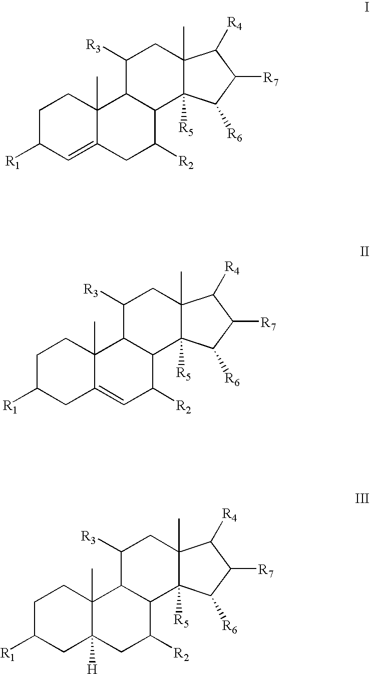
wherein R₁, R₂, R₃, R₄, R₅, R₆ may individually be chosen from hydrogen, OH, carbonyl, and an ascorbyl moiety; and R₇ may be hydrogen or any halogen.

42. A method for the treatment of diabetes which comprises administering to an animal in need of such treatment an anti-diabetic effective amount of a derivative having one or more of the following formulae: 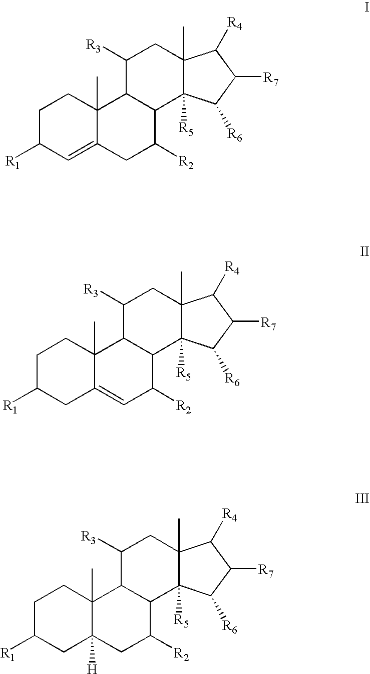
wherein R₁, R₂, R₃, R₄, R₅, R₆ may individually be chosen from hydrogen, OH, carbonyl, and an ascorbyl moiety; and R₇ may be hydrogen or any halogen.

43. A method for inhibiting weight gain in an animal which comprises administering to such animal a weight gain inhibiting amount of a derivative having one or more of the following formulae: 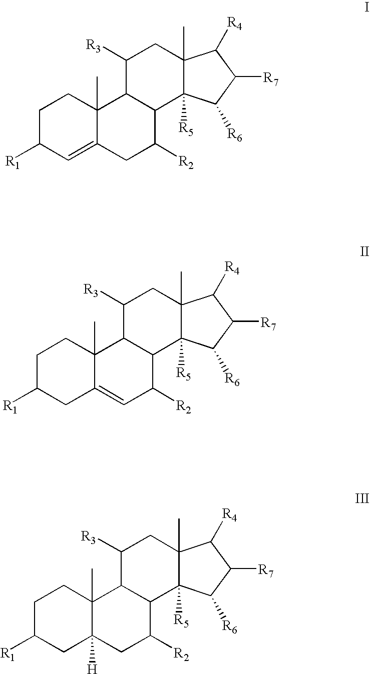
wherein R₁, R₂, R₃, R₄, R₅, R₆ may individually be chosen from hydrogen, OH, carbonyl, and an ascorbyl moiety; and R₇ may be hydrogen or any halogen.

44. A method of treating or preventing cardiovascular disease in an animal in need of such treatment or prevention which comprises administering a therapeutically effective amount of a derivative having one or more of the following formulae: 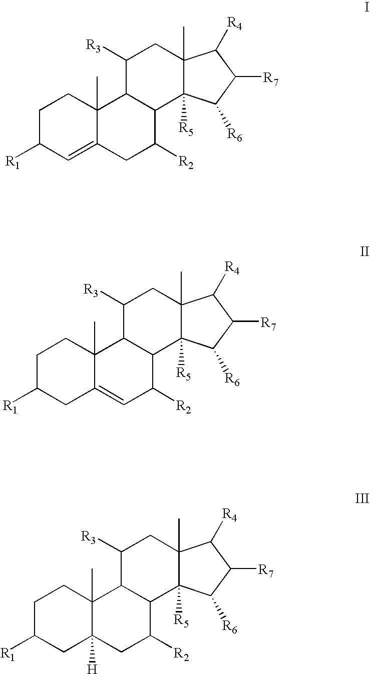
wherein R₁, R₂, R₃, R₄, R₅, R₆ may individually be chosen from hydrogen, OH, carbonyl, and an ascorbyl moiety; and R₇ may be hydrogen or any halogen.

45. A method of lowering serum cholesterol in an animal which comprises administering a therapeutically effective amount of a derivative having one or more of the following formulae: 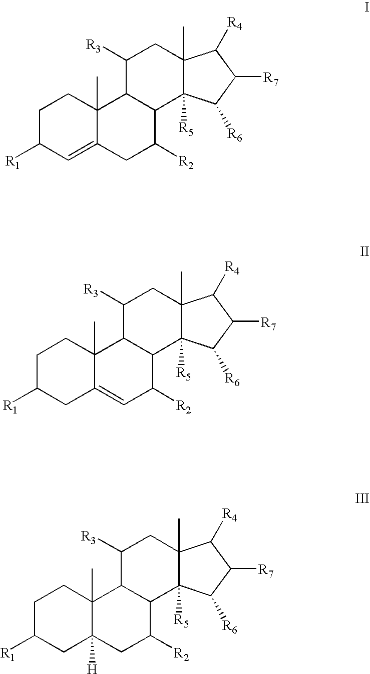
wherein R₁, R₂, R₃, R₄, R₅, R₆ may individually be chosen from hydrogen, OH, carbonyl, and an ascorbyl moiety; and R₇ may be hydrogen or any halogen.

46. A method of treating or preventing cancer in an animal in need of such treatment or prevention which comprises administering a therapeutically effective amount of a derivative having one or more of the following formulae: 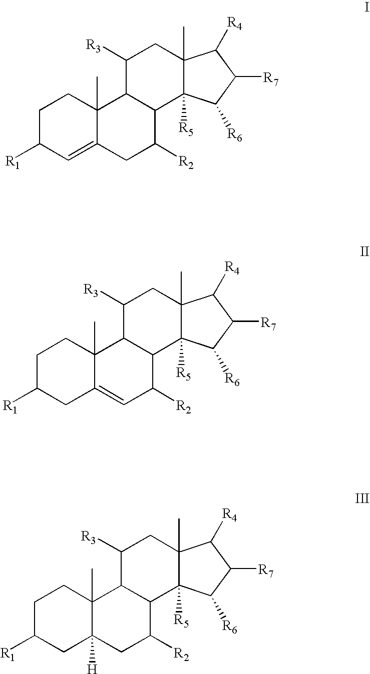
wherein R₁, R₂, R₃, R₄, R₅, R₆ may individually be chosen from hydrogen, OH, carbonyl, and an ascorbyl moiety; and R₇ may be hydrogen or any halogen.

47. A method of reducing inflammation in an animal in need of such reduction which comprises administering a therapeutically effective amount of a derivative having one or more of the following formulae: 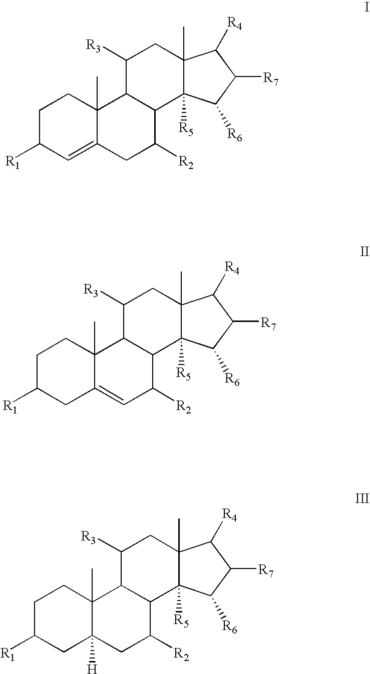
wherein R₁, R₂, R₃, R₄, R₅, R₆ may individually be chosen from hydrogen, OH, carbonyl, and an ascorbyl moiety; and R₇ may be hydrogen or any halogen.
